(19) United States
(12) Patent Application Publication
(10) Pub. No.: US 2024/0360349 A1
(43) Pub. Date: Oct. 31, 2024

(54) IONIC LIQUID AND METHOD FOR PRODUCING THE SAME

(71) Applicant: Nisshinbo Holdings Inc., Tokyo (JP)

(72) Inventors: Gen Masuda, Chiba-city (JP); Tatsuya Ochikubo, Chiba-city (JP)

(73) Assignee: Nisshinbo Holdings Inc., Tokyo (JP)

(21) Appl. No.: 18/681,652

(22) PCT Filed: Jul. 14, 2022
(86) PCT No.: PCT/JP2022/027664

§ 371 Date: Feb. 6, 2024

(30) Foreign Application Priority Data
Aug. 17, 2021 (JP) 2021-132896

(51) Int. Cl. C09K 3/00

(52) U.S. Cl. CPC C09K 3/00

(57) ABSTRACT

Provided is an ionic liquid that can be used for semiconductor manufacturing and that has a low metal ion content. This ionic liquid comprises cations and onions and contains, each by not more than 100 ppb, metal ions of 16 elements indicated by the following element symbols: Li, Na, Ca, Mg, Al, K, Ti, Cr, Mu, Fe, Co, Ni, Cu, Zn, Ba, and W.

### TECHNICAL FIELD

The present invention relates to an ionic liquid and to a method for producing the same. More specifically, the invention relates to a high-purity ionic liquid having a reduced metal ion content and to a method for producing such an ionic liquid.

### BACKGROUND ART

Ionic liquids, which are salts composed only of ions, are attracting interest because of their outstanding characteristics, including non-volatility, flame retardancy and high ionic conductivity, and also because the properties and functionalities of ionic liquids can be variously designed. By virtue of these qualities, ionic liquids show promise in such applications as solvents in environmentally friendly green chemistry and electrolytes in electrical storage devices.

However, on account of their physical properties, ionic liquids are impossible to purify by distillation or recrystallization, and so are known to be substances for which achieving a higher level of purity is difficult.

For example, Patent Documents 1 and 2 disclose high-purity bis (fluorosulfonyl) imide salts in which the fluoride ion content has been reduced to 100 ppm or less. Yet, high-purity ionic liquids with low enough metal ion contents to enable use in semiconductor fabrication processes which do not tolerate metal ion contamination, and methods for producing such ionic liquids, are not yet known.

### PRIOR ART DOCUMENTS

### Patent Documents

Patent Document 1: JP-A 2013-199480
Patent Document 2: JP-A 2014-40369

### SUMMARY OF INVENTION

### Technical Problem

In light of such circumstances, the objects of this invention are to provide an ionic liquid which has a low content of metal ions and can be used in semiconductor manufacturing processes, to provide a method for producing the same, and also to provide a novel ionic liquid.

### Solution to Problem

The inventors have conducted intensive investigations aimed at achieving the above objects. As a result, they have discovered an ionic liquid that has low contents of specific metallic species and a method for producing such an ionic liquid.

Accordingly, the invention provides:

1. An ionic liquid composed of cations and anions, wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less:
2. The ionic liquid of 1 above, wherein metal ions of said 16 elements are all included in respective amounts of 10 ppb or less;
3. The ionic liquid of 1 above, wherein metal ions of the 26 elements indicated by the symbols Li, Na, Ca. Mg, Al, K, Ti, Cr, Mn, Fe, Co, Ni, Cu, Zn, Ba, W, V. Sr, Zr, Mo, Ag, Cd. Ta. TI, Pb and Bi are all included in respective amounts of 100 ppb or less:
4. The ionic liquid of 3 above, wherein metal ions of said 26 elements are all included in respective amounts of 10 ppb or less;
5. The ionic liquid of any of 1 to 4 above, wherein the cations are monovalent cations selected from the group consisting of quaternary ammonium ions, pyridinium ions, cyclic amidinium ions and quaternary phosphonium ions;
6. The ionic liquid of any of 1 to 5 above, wherein the anions are monovalent anions selected from the group consisting of trialkylsilyl group-containing alkyl sulfonate ions, tetrafluoroborate ion, alkyl sulfate ions, hexafluorophosphate ion, bis (trifluoromethanesulfonyl) amide ion, bis (fluorosulfonyl) amide ion, alkyl sulfonate ions, aryl sulfonate ions, trifluoromethanesulfonate ion, acetate ion and alkyl phosphate ions;
7. The ionic liquid of any one of 1 to 6 above being used in a semiconductor fabrication process;
8. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method includes the step of contacting an ionic liquid composed of cations and anions with a chelating resin material;
9. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method includes the step of alkylating a tertiary alkylamine or an N-alkylimidazole with a dialkyl sulfate, an alkyl sulfonate, an aryl sulfonate, an alkyl trifluoromethanesulfonate or a trialkyl phosphate;
10. The ionic liquid production method of 9 above, which method includes the step of using, as the tertiary alkylamine or N-alkylimidazole and as the dialkyl sulfate, alkyl sulfonate, aryl sulfonate, alkyl trifluoromethanesulfonate, alkyl trifluoromethanesulfonate or trialkyl phosphate, compounds which contain metal ions of the 16 elements indicated by the symbols Li, Na, Ca, Mg, Al, K, Ti, Cr, Mn, Fe, Co. Ni, Cu, Zn, Ba and W in respective amounts of 100 ppb or less;
11. A method for producing, by neutralization of an acid and a base, an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method includes the step of using, as the acid and the base, compounds which contain metal ions of said 16 elements in respective amounts of 100 ppb or less;
12. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method includes the steps of, in order: contacting a salt of a quaternary cation salt with a chelating resin material so as to remove metal ions included within the salt, converting the anion of the resulting quaternary cation salt to a hydroxide ion by means of an anion exchange resin, and mixing together equivalent amounts of the salt converted to a hydroxide ion and a proton adduct of an anion that gives the desired ionic liquid to effect neutralization;
13. The ionic liquid production method of any of 8 to 12 above, wherein metal ions of said 16 elements are all included in respective amounts of 10 ppb or less;
14. The ionic liquid production method of any of 8 to 12 above, wherein metal ions of the 26 elements indicated by the symbols Li, Na, Ca, Mg, Al, K, Ti, Cr, Mn, Fe, Co, Ni, Cu, Zn, Ba, W, V, Sr, Zr, Mo, Ag, Cd, Ta, TI, Pb and Bi are all included in respective amounts of 100 ppb or less;
15. The ionic liquid production method of 14 above, wherein metal ions of said 26 elements are all included in respective amounts of 10 ppb or less;
16. An ionic liquid of formula (A) below

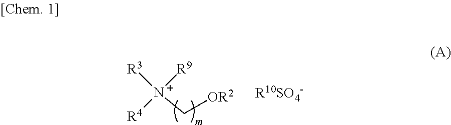

(wherein R³, R⁴ and R⁹ are each independently an alkyl group of 1 to 3 carbon atoms, R¹⁰ is an alkyl group of 1 to 4 carbon atoms, R² is a methyl group or an ethyl group, and m is the integer 1 or 2);
17. The ionic liquid according to 16 above of formula (A1) below

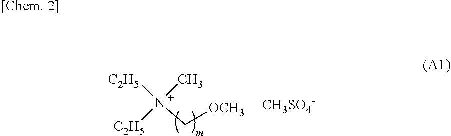

(wherein m is the integer 1 or 2);
18. The ionic liquid of formula (B) below

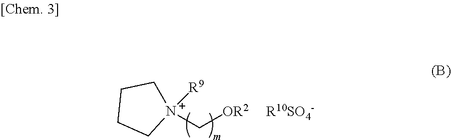

(wherein R⁹ is an alkyl group of 1 to 3 carbon atoms, R² is a methyl group or an ethyl group, R¹⁰ is an alkyl group of 1 to 4 carbon atoms, and m is the integer 1 or 2);
19. The ionic liquid according to 18 above of formula (B1) below

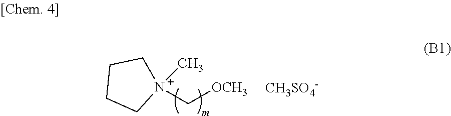

(wherein m is the integer 1 or 2);
20. The ionic liquid of formula (C) below

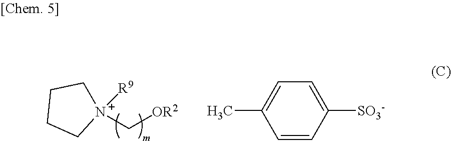

(wherein R⁹ is an alkyl group of 1 to 3 carbon atoms, R² is a methyl group or an ethyl group, and m is the integer 1 or 2); and
21. The ionic liquid of 20 above, wherein R² is a methyl group and R⁹ is a methyl group or an ethyl group.

### Advantageous Effects of Invention

The ionic liquid of the invention, because it contains only low levels of given metal ions and has a high purity, can be used even in semiconductor fabrication processes.

### BRIEF DESCRIPTION OF DRAWINGS

FIG. 1 is a 1H-NMR spectrum of Ionic Liquid [3] obtained in Example 2-1.

FIG. 2 is a ¹H-NMR spectrum of Ionic Liquid [4] obtained in Example 2-2.

FIG. 3 is a 1H-NMR spectrum of Ionic Liquid obtained in Example 2-9.

FIG. 4 is a 1H-NMR spectrum of Ionic Liquid obtained in Example 2-10.

### DESCRIPTION OF EMBODIMENTS

This invention is described more fully below.

The ionic liquid according to the invention is composed of cations and anions and includes metal ions of the 16 elements indicated by the symbols Li, Na, Ca, Mg, Al, K, Ti, Cr, Mn, Fe, Co, Ni, Cu, Zn, Ba and W, preferably metal ions of the 26 elements indicated by the symbols Li, Na, Ca, Mg, Al, K, Ti, Cr, Mn, Fe, Co, Ni, Cu, Zn, Ba, W, V, Sr, Zr, Mo, Ag, Cd, Ta, Tl, Pb and Bi, in respective amounts of 100 ppb or less, preferably 10 ppb or less.

As mentioned later in the “Examples” section, the metal contents are analytical values obtained by inductively coupled plasma mass spectrometry (ICP-MS).

While it acceptable for the ionic liquid to be an ionic liquid composed of known cations and anions, an ionic liquid in which the cations are monovalent cations selected from the group consisting of quaternary ammonium ions, pyridinium ions, cyclic amidinium ions and quaternary phosphonium ions is preferred.

Examples of quaternary ammonium ions include tetraalkylammonium ions and tetraalkylammonium ions having alkoxy-substituted alkyl groups. Ammonium ions having alkyl groups and alkoxy-substituted alkyl groups on the nitrogen atom are preferred. Quaternary ammonium ions of formula (1) and pyrrolidinium ions of formula (2) are more preferred.

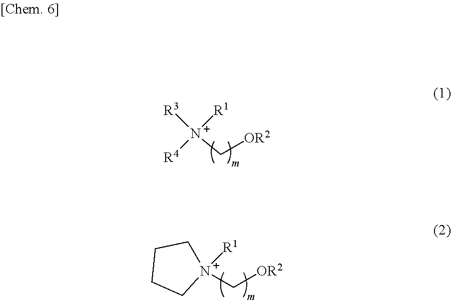

In these formulas, R¹ is a hydrogen atom or an alkyl group of 1 to 3 carbon atoms, R³ and R⁴ are each independently an alkyl group of 1 to 3 carbon atones, R² is a methyl group or an ethyl group, and m is the integer 1 or 2.

Examples of alkyl groups of 1 to 3 carbon atoms include methyl, ethyl and n-propyl groups. Ethyl and methyl groups are especially preferred. It is more preferable for R¹ to be a methyl group and for R³ and R⁴ to be ethyl groups, or for R¹ and R³ to be methyl groups and for R⁴ to be an ethyl group.

Examples of cyclic amidinium ions include imidazolinium ions, diazabicycloundecene ions and diazabicyclononene ions.

Examples of imidazolinium ions include 1-alkyl-3-alkylimidazolinium ions. Imidazolinium ions of formula (3) are preferred.

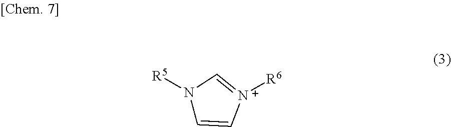

In formula (3), R⁶ is a hydrogen atom or an alkyl group of 1 to 8 carbon atoms, and R⁵ is an alkyl group of 1 to 8 carbon atoms.

The alkyl group of 1 to 8 carbon atoms may be linear, branched or cyclic. Examples include methyl, ethyl, n-propyl, i-propyl, c-propyl, n-butyl, i-butyl, s-butyl, t-butyl, c-butyl, n-pentyl, c-pentyl, n-hexyl, c-hexyl, n-heptyl and n-octyl groups.

Of these, R⁶ is preferably a hydrogen atom or an alkyl group of 1 to 4 carbon atoms, and more preferably a hydrogen atom or a methyl, ethyl, n-propyl or n-butyl group; and R⁵ is preferably an alkyl group of 1 to 4 carbon atoms, and more preferably a methyl or ethyl group.

Exemplary diazabicycloundecene ions include those of formula (4). Exemplary diazabicyclononene ions include those of formula (5) below.

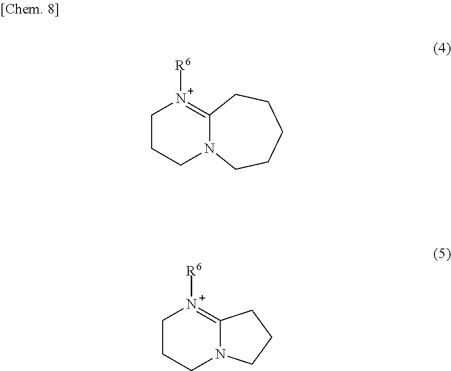

In these formulas, R⁶ has the same meaning as described above.

Exemplary pyridinium ions include those of formula (6). Specific examples include the N-propylpyridinium, N-butylpyridinium, 1-butyl-4-methylpyridinium and 1-butyl-2,4-dimethylpyridinium ions.

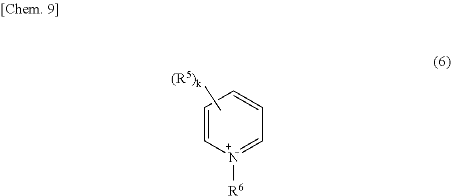

In the formula, R⁵ and Re have the same meanings as above, and k is an integer from 1 to 5.

Exemplary quaternary phosphonium ions include tetraalkylphosphonium ions and trialkylalkoxyphosphonium ions, although phosphonium ions of formula (7) are preferred.

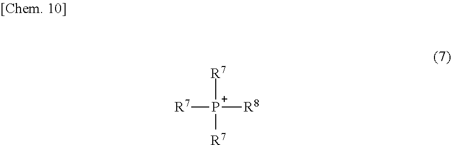

In formula (4), R′ is an alkyl group of 1 to 30 carbon atoms, and R⁸ is an alkyl group or alkoxy group of 1 to 30 carbon atoms.

The alkyl group of 1 to 30 carbon atoms may be linear, branched or cyclic. Examples include methyl, ethyl, n-propyl, i-propyl, c-propyl, n-butyl, i-butyl, s-butyl, t-butyl, c-butyl, n-pentyl, c-pentyl, n-hexyl, c-hexyl, n-heptyl, n-octyl, 2-ethylhexyl, n-nonyl, n-decyl, n-undecyl, n-dodecyl, n-tridecyl, n-tetradecyl, u-pentadecyl, n-hexadecyl, n-heptadecyl, n-octadecyl n-nonadecyl and n-eicosyl groups.

Examples of the alkoxy group of 1 to 30 carbon atoms, in which the alkyl group may be linear, branched or cyclic, include methoxy, ethoxy, n-propoxy, i-propoxy, c-propoxy, n-butoxy, i-butoxy, s-butoxy, t-butoxy, c-butoxy, n-pentyloxy, c-pentyloxy, n-hexyloxy, c-hexyloxy, n-heptyloxy, n-octyloxy, 2-ethylhexyloxy, n-nonyloxy, n-decyloxy, n-undecyloxy, n-dodecyloxy, n-tridecyloxy, n-tetradecyloxy, n-pentadecyloxy, u-hexadecyloxy, n-heptadecyloxy, n-octadecyloxy, n-nonadecyloxy and n-eicosyloxy groups.

Of these, R⁷ is preferably a linear alkyl group of 2 to 8 carbon atoms, and more preferably a linear alkyl group of 2 to 6 carbon atoms. From the standpoint of the availability of the starting materials, n-ethyl, n-butyl and n-hexyl groups are more preferred. Even among these, from the standpoint of the ease of forming an ionic liquid, n-butyl and n-hexyl groups are especially preferred.

R⁸ is preferably a linear alkyl group of 10 to 20 carbon atoms, and more preferably a linear alkyl group of 12 to 20 carbon atoms.

As for the anions, they may be selected from among known anions capable of forming an ionic liquid with the various above-described cations. Examples include tetrafluoroborate ion (BF₄⁻), BF(CN)₃⁻, BF₃CF₃⁻, BF: (CF₂CF₃)⁻, hexafluorophosphate ion (PF₆⁻), bis (trifluoromethanesulfonyl) amide (TFSA) ion, bis (fluorosulfonyl) amide (FSA) ion, alkyl sulfonate ions, aryl sulfonate ions, trifluoromethanesulfonate ion, trialkylsilyl group-containing alkyl sulfonate ions, alkyl sulfate anions, alkyl phosphate anions, alkyl phosphite anions, amino acid anions, carboxylate anions, Cl⁻, Br⁻, I⁻, nitrate anion and saccharinate anion. Of these, BF₄⁻, trialkylsilyl group-containing alkyl sulfonate ions, alkyl sulfate ions, alkyl phosphate ions, aryl sulfonate ions, trifluoromethanesulfonate ion and carboxylate anions are preferred.

Examples of alkyl sulfonate ions include methanesulfonate and ethanesulfonate anions. Examples of aryl sulfonate ions include benzenesulfonate and p-toluenesulfonate anions.

Examples of alkyl sulfate ions include those of formula (8) below. Examples of alkyl phosphate ions include those of formula (9) below. Examples of trialkylsilyl group-containing alkyl sulfonate ions include those of formula (10) below.

Examples of carboxylate anions include formate anion and acetate anion.

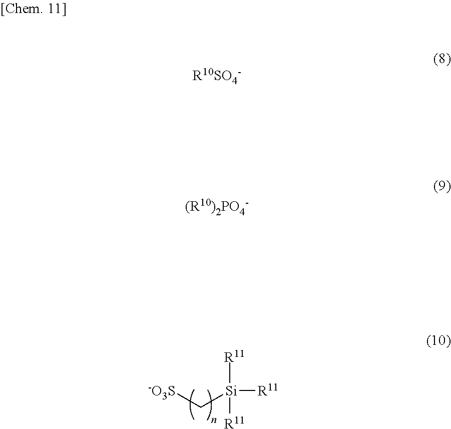

In formulas (8) and (9), each R¹⁰ is independently an alkyl group of 1 to 4 carbon atoms. This alkyl group is exemplified by those groups mentioned above as examples of R⁶ which have from 1 to 4 carbon atoms. Methyl and ethyl groups are preferred.

In formula (10), each R¹¹ is independently an alkyl group of 1 to 8 carbon atom, and n is an integer from 2 to 8, preferably an integer from 2 to 6. This alkyl group is exemplified by the same groups mentioned above as examples of R⁶, although alkyl groups of 1 to 3 carbon atoms are preferred, and a methyl group is more preferred.

The above ionic liquids can be produced by known methods, and some may be acquired as commercial products.

An exemplary method for lowering the various metallic elements mentioned above to or below the prescribed amounts and thereby raising the purity of the ionic liquid involves bringing an ionic liquid synthesized by a known method or acquired as a commercial product into contact with a chelating resin material.

The method for contacting the ionic liquid with a chelating resin is not particularly limited, and may consist of batch treatment in which a chelating resin is added to the ionic liquid, column treatment in which the ionic liquid is passed through a column packed with a chelating resin, filtration in which the ionic liquid is passed through a chelating resin filter, or combinations of these techniques.

Also, the number of times that the ionic liquid is contacted with the chelating resin is not particularly limited, so long as the metal contents are brought down to the desired level, and may be one time only or may be a plurality of two or more times.

Examples of chelating resins include iminodiacetic acid-based chelating resins, aminophosphoric acid-based chelating resins and polyamine-based chelating resins. These may each be used singly or two or more may be used together.

These chelating resins can be acquired as commercial products. Examples include the chelating resins ORLITE DS-22 and ORLITE DS-21, the iminodiacetic acid-based chelating resin Ambersep IRC748 and the aminophosphoric acid-based chelating resin Ambersep IRC747UPS, all from Organo Corporation; and the iminodiacetic acid-based chelating resin Diaion CR11 and the polyamine-based chelating resin Diaion CR20, both from Mitsubishi Chemical Corporation.

When treating the ionic liquid with a chelating resin, to improve handleability due to a decrease in viscosity and otherwise increase treatment efficiency, treatment may be carried out using a solution of the ionic liquid dissolved in a solvent.

Examples of the solvent include water and the following organic solvents: alcohols such as methanol and ethanol; acyclic ethers such as dibutyl ether, 1,2-dimethoxyethane, 1,2-ethoxymethoxyethane, methyl diglyme, methyl triglyme, methyl tetraglyme, ethyl glyme, ethyl diglyme, butyl diglyme, ethyl cellosolve, ethyl carbitol, butyl cellosolve and butyl carbitol; heterocyclic ethers such as tetrahydrofuran, 2-methyltetrahydrofuran, 1,3-dioxolane and 4,4-dimethyl-1,3-dioxane; lactones such as γ-butyrolactone, γ-valerolactone, δ-valerolactone. 3-methyl-1,3-oxazolidin-2-one and 3-ethyl-1,3-oxazolidin-2-one; amides such as N-methylformamide, N,N-dimethylformamide, N-methylacetamide and N-methylpyrrolidinone; carbonates such as diethyl carbonate, dimethyl carbonate, ethyl methyl carbonate, propylene carbonate, ethylene carbonate and butylene carbonate; imidazolines such as 1,3-dimethyl-2-imidazolidinone; and nitriles such as acetonitrile and propionitrile. These may be used singly or two or more may be used in admixture.

In this case, by treating the purified solution under heating, under a vacuum or under both of these conditions, it is possible to remove the solvent used, enabling a high-purity ionic liquid in terms of residual content to be obtained.

In the case of ionic liquids having the above-mentioned alkyl sulfate ions, alkyl sulfonate ions, aryl sulfonate ions, trifluoromethanesulfonate anion or alkyl phosphate ions, a high-purity ionic liquid in which the contents of metal ions of the various metallic elements mentioned above are low can be obtained by synthesizing the ionic liquid via the so-called acid ester process in which a tertiary alkyl amine or N-alkyl imidazole that has been highly purified beforehand via an existing process that removes metal ions of the above-mentioned 16 elements, preferably 26 elements, to contents of 100 ppb or less, preferably 10 ppb or less, is alkylated using as the alkylating agent dialkyl sulfate, alkyl sulfonate, aryl sulfonate, perfluorosulfonate such as ethyl trifluoromethanesulfonate or trialkyl phosphate that has been highly purified beforehand by an existing process that removes metal ions of the above-mentioned 16 elements, preferably 26 elements, to contents of 100 ppb or less, preferably 10 ppb or less.

Known conditions mentioned in, for example. Ion Ekitai—J̅oshiki wo Kutsugaesu Fushigi na Shio—[Ionic liquids—Amazing salts which upend conventional wisdom], pp. 8-9 (2005, Corona Publishing) can be employed as the conditions for the acid ester process. For example, use can be made of a method that mixes an amine starting material such as a tertiary alkyl amine with an alkylating agent under ice cooling in a 1:1 molar ratio, raises the temperature to room temperature, stirs the system for about 2 hours and then purifies under reduced pressure and applied heat for about 1 hour.

Suitable examples of ionic liquids synthesized by the acid ester process include, but are not limited to, those having the following formulas.

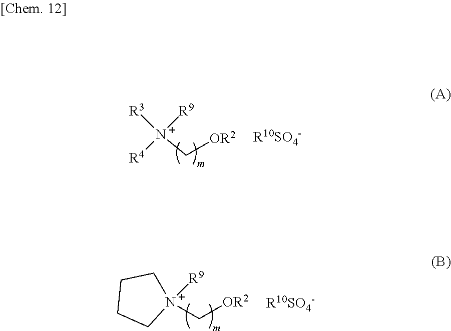

In these formulas, R³, R⁴ and R⁹ are each independently an alkyl group of 1 to 3 carbon atoms, R¹⁰ is an alkyl group of 1 to 4 carbon atoms, R² is a methyl or ethyl group, and m is the integer 1 or 2. The alkyl group of 1 to 3 carbon atoms is exemplified by the same groups mentioned above as examples of R¹. The alkyl group of 1 to 4 carbon atoms is exemplified by those groups of 1 to 4 carbons among the groups mentioned above as examples of R⁶.

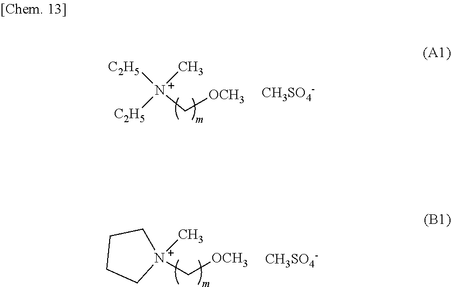

In these formulas, m is the integer 1 or 2, with 2 being preferred.

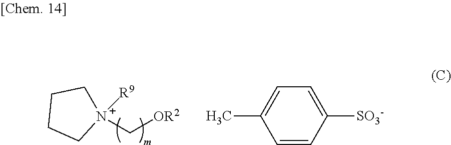

In this formula, R², R⁹ and m are as defined above.

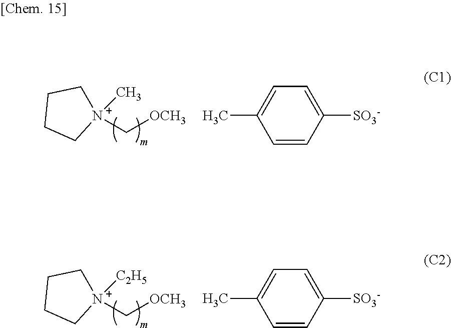

In these formulas, m is the integer 1 or 2, with 2 being preferred.

High-purity ionic liquids can be obtained also by a production method that employs the so-called neutralization process, which is a process for producing an ionic liquid by the neutralization of an acid and a base.

When producing a conventional quaternary salt-based ionic liquid by the neutralization process, the ionic liquid can be obtained by dissolving, for example, a halide salt of the quaternary cation that gives the target ionic liquid in water or an organic solvent capable of dissolving the salt, then converting the anions such as halide ions into hydroxide ions using an anion exchange resin and mixing together equivalent amounts of the resulting salt converted to hydroxide ions and, as the anion source, a proton adduct (acid) of the desired anion to effect neutralization, and subsequently removing, such as by drawing a vacuum, by-product water, solvent water and, in cases where an organic solvent is used as the solvent, the organic solvent. When producing a high-purity ionic liquid in this way, it is desirable to first treat the halide salt or the like of the quaternary cations with, for example, the above-described chelating resin so as remove the metals down to a metal ion content for the above-mentioned 16 elements, preferably 26 elements, of 100 ppb or less, preferably 10 ppb or less, and carry out ion exchange using water or another solvent (e.g., a semiconductor grade solvent) of sufficiently high purity.

The anionic exchange resin is not particularly limited and may be suitably selected and used from among known anionic exchange resins.

In the case of neutral salt-based ionic liquids, a high-purity ionic liquid having low contents of metal ions of the various above-mentioned metallic elements can be obtained by synthesis using as the starting materials a base such as an amine and an acid such as an organic acid that have been highly purified by a known purification process such as distillation and, if necessary, treated with the above-described chelating resin material to remove metal ions of the above-mentioned 16 elements, preferably 26 elements, down to contents of 100 ppb or less, preferably 10 ppb or less.

As used herein, “neutral salt-based ionic liquid” refers to an ionic liquid which is composed of a salt obtained by an acid-base neutralization reaction (see Ion-sei Ekitai—Kathatsu no Saizensen to Mirai—[Ionic liquids—The leading edge of research and future prospects]. pp. 19-21. CMC Shuppan (2003)) and which has cations formed by the addition of protons.

A still higher level of purity may be achieved by bringing a high-purity ionic liquid obtained by the acid ester process or the neutralization process into contact with the above-described chelating resin.

When carrying out the ionic liquid production method of the invention, non-metallic members such as glass members, plastic members, Teflon®-coated members or fluoroplastic-coated members are used as those portions of the reactor, columns, tubing and the like which come into contact with the ionic liquid or ionic liquid solution. Plastic members, Teflon®-coated members and fluoroplastic-coated members are especially preferred.

As explained above, because the contents of given metal ions in the ionic liquid of the invention are reduced to 100 ppb or less by chelating resin treatment and synthesis via the acid ester process or the neutralization process, the ionic liquid can be suitably used in semiconductor fabrication processes which do not tolerate metal ion contamination.

### EXAMPLES

Examples and Comparative Examples are given below by way of illustration, although the invention is not limited by these Examples.

The analytical equipment and conditions used were as follows.

### (1) 1H-NMR Spectrum

JNM-ECZ400S, from JEOL, Ltd.

### (2) Metal Ion Measurement

### [Pre-Treatment]

The sample was wet decomposed, following which the liquid obtained by acid dissolution of the residue was used as the test liquid to be measured. Sample preparation was carried out in a clean draft chamber (Class 10) installed within a clean room (Class 100).

### [Measurement]

Measurement was carried out by induction-coupled plasma mass spectrometry (ICP-MS).

### [Calculation of Analytical Values]

Impurity concentrations (ng/g) were calculated by dividing the measured element weights (ng) by the weight of the analyzed sample (g).

### [1] Chelating Resin Treatment

### Example 1-1

ICP-MS measurement of the ionic liquid of formula [1] below was carried out, whereupon, as shown in Table 1, Na, Ca and Zr were found to be present in amounts greater than 10 ppb. A 25.0 g amount of this compound was dissolved in 5.0 g of methanol (semiconductor grade; Kanto Chemical Co., Inc.). This solution was passed three times through a plastic column (eluant, acetonitrile) packed with 5.0 g (7.5 mL) of the chelating resin ORLITE DS-22 (Organo Corporation). The resulting eluate was collected in a sterilized polypropylene centrifuge tube “mini” (IWAKI) container and concentrated by applying a vacuum. This concentrate was subjected to ICP-MS measurement, as a result of which the contents of the 26 elements were all at or below the lower limit of determination (at or below 100 ppb or 10 ppb). Because the ionic liquid of formula [1] below contains phosphorus within the structure and interference elimination thereof to a sufficient degree is impossible, the lower limit of determination for some elements (Li, K, Ti, V, Cu) is 100 ppb.

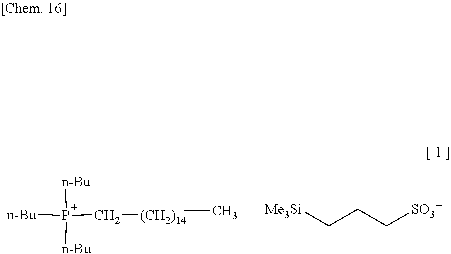

### Example 1-2

ICP-MS measurement of the ionic liquid of formula [2] below was carried out, whereupon, as shown in Table 1, Na, Al, K and Zr were found to be present in amounts greater than 10 ppb. A 25.0 g amount of this compound was dissolved in 5.0 g of acetonitrile (LC/MS grade, Honeywell). This solution was passed three times through a plastic column (eluant, acetonitrile) packed with 5.0 g (7.5 mL) of the chelating resin ORLITE DS-22 (Organo Corporation). The resulting eluate was collected in a sterilized polypropylene centrifuge tube “mini” (IWAKI) container and concentrated by applying a vacuum. This concentrate was subjected to ICP-MS measurement, as a result of which the contents of the 26 elements were all 10 ppb or less.

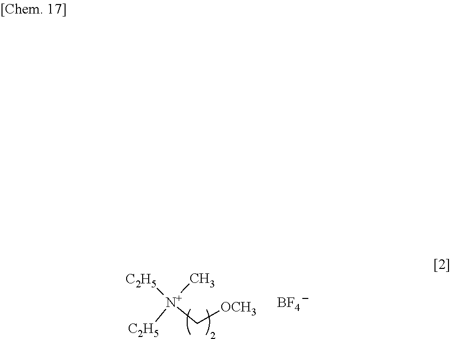

### [2] Acid Ester Process

### Example 2-1

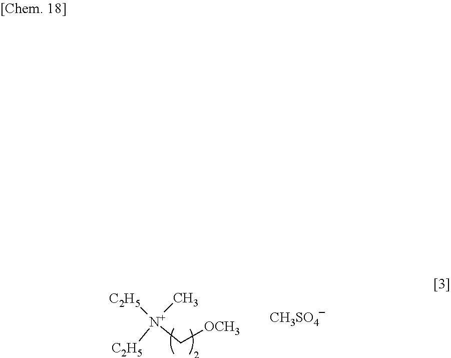

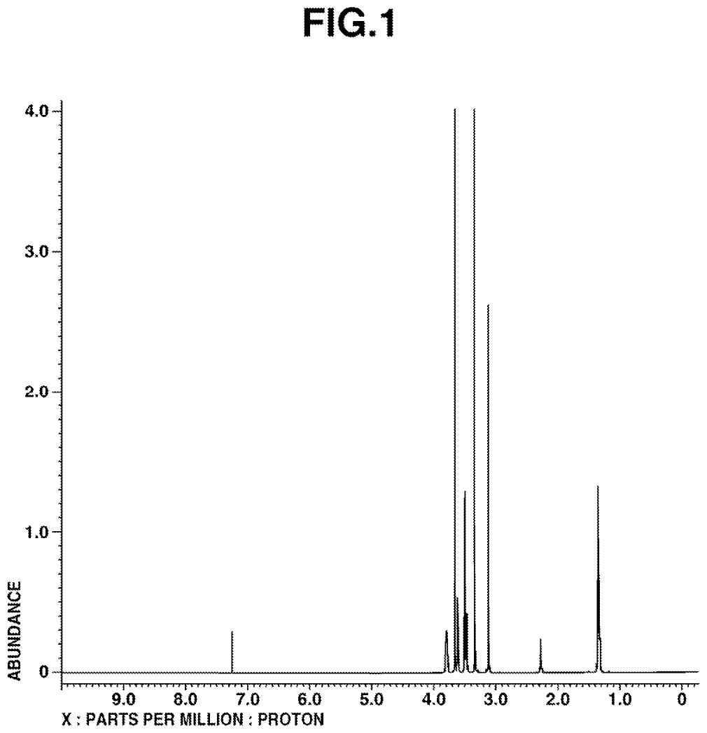

N,N-Diethyl-N-2-methoxyethylamine synthesized by an existing method and dimethyl sulfate (Kanto Chemical Co., Inc.) were precisely distilled and reacted for 2 days at 100° C. under hermetically sealed conditions in a glass autoclave. A vacuum was then applied for 3 hours and the unreacted starting materials were removed by distillation, giving Ionic Liquid [3]. ICP-MS measurement of this ionic liquid was carried out, whereupon, as shown in Table 1, both Na and K were found to be present in amounts greater than 10 ppb. By carrying out chelating resin treatment in the same way as in Example 1-1, an ionic liquid was obtained in which the contents of the 26 elements as determined by ICP-MS measurement were all 10 ppb or less. FIG. 1 shows the 1H-NMR spectrum (solvent, heavy chloroform) of Ionic Liquid [3].

TABLE 1

| | | Li | Na | Mg | Al | K | Ca | Ti | V | Cr | Mn | Fe | Co | Ni |
|---|---|---|---|---|---|---|---|---|---|---|---|---|---|---|
| Exam- | Before | <100 | 40 | <10 | <10 | <100 | 400 | <100 | <100 | <10 | <10 | <10 | <10 | <10 |
| ple | treat- | | | | | | | | | | | | | |
| 1-1 | ment | | | | | | | | | | | | | |
| | After | <100 | <10 | <10 | <10 | <100 | <10 | <100 | <100 | <10 | <10 | <10 | <10 | <10 |
| | treat- | | | | | | | | | | | | | |
| | ment | | | | | | | | | | | | | |
| Exam- | Before | <10 | 2,000 | <10 | 20 | 100 | <10 | <10 | <10 | <10 | <10 | <10 | <10 | <10 |
| ple | treat- | | | | | | | | | | | | | |
| 1-2 | ment | | | | | | | | | | | | | |
| | After | <10 | <10 | <10 | <10 | <10 | <10 | <10 | <10 | <10 | <10 | <10 | <10 | <10 |
| | treat- | | | | | | | | | | | | | |
| | ment | | | | | | | | | | | | | |
| Exam- | Before | <10 | 400 | <10 | <10 | 10 | <10 | <10 | <10 | <10 | <10 | <10 | <10 | <10 |
| ple | treat- | | | | | | | | | | | | | |
| 2-1 | ment | | | | | | | | | | | | | |
| | After | <10 | <10 | <10 | <10 | <10 | <10 | <10 | <10 | <10 | <10 | <10 | <10 | <10 |
| | treat- | | | | | | | | | | | | | |
| | ment | | | | | | | | | | | | | |

| | | Cu | Zn | Sr | Zr | Mo | Ag | Cd | Ba | Ta | W | Tl | Pb | Bi |
|---|---|---|---|---|---|---|---|---|---|---|---|---|---|---|
| Exam- | Before | <100 | 80 | <10 | <10 | <10 | <10 | <10 | <10 | <10 | <10 | <10 | <10 | <10 |
| ple | treat- | | | | | | | | | | | | | |
| 1-1 | ment | | | | | | | | | | | | | |
| | After | <100 | <10 | <10 | <10 | <10 | <10 | <10 | <10 | <10 | <10 | <10 | <10 | <10 |
| | treat- | | | | | | | | | | | | | |
| | ment | | | | | | | | | | | | | |
| Exam- | Before | <10 | <10 | <10 | 10 | <10 | <10 | <10 | <10 | <10 | <10 | <10 | <10 | <10 |
| ple | treat- | | | | | | | | | | | | | |
| 1-2 | ment | | | | | | | | | | | | | |
| | After | <10 | <10 | <10 | <10 | <10 | <10 | <10 | <10 | <10 | <10 | <10 | <10 | <10 |
| | treat- | | | | | | | | | | | | | |
| | ment | | | | | | | | | | | | | |
| Exam- | Before | <10 | <10 | <10 | <10 | <10 | <10 | <10 | <10 | <10 | <10 | <10 | <10 | <10 |
| ple | treat- | | | | | | | | | | | | | |
| 2-1 | ment | | | | | | | | | | | | | |
| | After | <10 | <10 | <10 | <10 | <10 | <10 | <10 | <10 | <10 | <10 | <10 | <10 | <10 |
| | treat- | | | | | | | | | | | | | |
| | ment | | | | | | | | | | | | | |

### Example 2-2

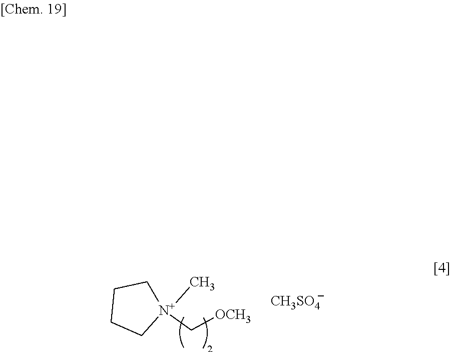

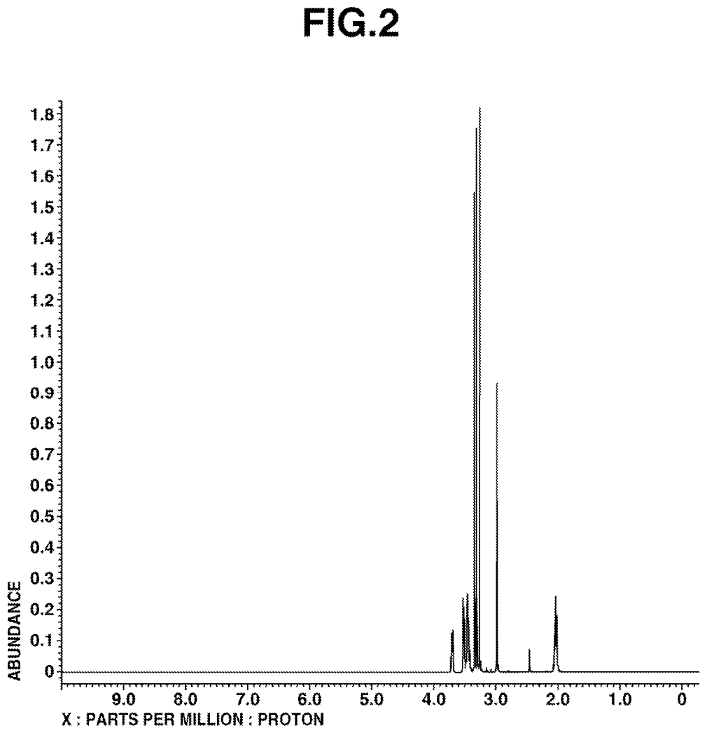

N-2-Methoxyethylpyrrolidine synthesized by an existing method and dimethyl sulfate (Kanto Chemical Co., Inc.) were each precisely distilled, following which they were each treated with the same weight of the chelating resin ORLITE DS-22, charged into a Teflon®-coated autoclave in equimolar amounts, and reacted at 100° C. for 2 hours under hermetically sealed conditions. A vacuum was then applied for 3 hours and the unreacted starting materials were removed by distillation, giving Ionic Liquid [4]. The contents of the 26 elements in Ionic Liquid [4] as determined by ICP-MS measurement were all 10 ppb or less. FIG. 2 shows the 1H-NMR spectrum (solvent, heavy chloroform) of Ionic Liquid [4].

### Example 2-3

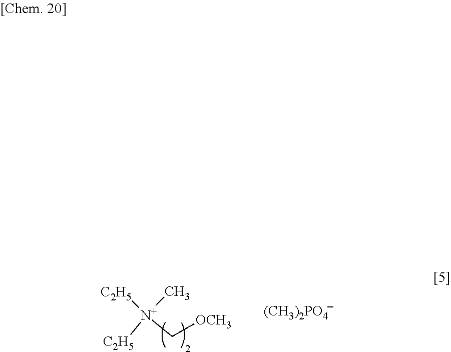

Aside from changing the starting materials to N,N-diethyl-N-2-methoxyethylamine and trimethyl phosphate (FUJIFILM Wako Pure Chemical Corporation), Ionic Liquid [5] in which the contents of the 26 elements as determined by ICP-MS measurement were all at or below the lower limit of determination (at or below 100 ppb or 10 ppb) was obtained in the same way as in Example 2-2. Because Ionic Liquid [5] contains phosphorus within the structure and interference elimination thereof to a sufficient degree is impossible, the lower limit of determination for some elements (Li, K, Ti, V, Cu) is 100 ppb.

### Example 2-4

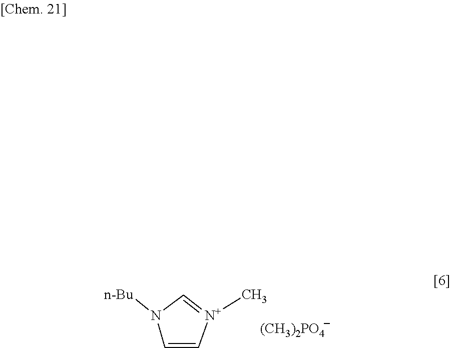

Aside from changing the starting materials to 1-butylimidazole (Kanto Chemical Co., Ltd.), Ionic Liquid [6] in which the contents of the 26 elements as determined by ICP-MS measurement were all at or below the lower limit of determination (at or below 100 ppb or 10 ppb) was obtained in the same way as in Example 2-3. Because Ionic Liquid [6] contains phosphorus within the structure and interference elimination thereof to a sufficient degree is impossible, the lower limit of determination for some elements (Li, K, Ti, V, Cu) is 100 ppb.

### Example 2-5

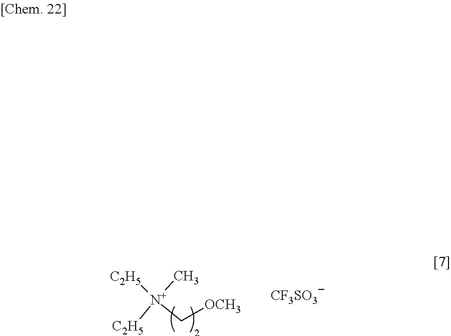

Aside from changing one of the starting materials from dimethyl sulfate to methyl trifluoromethanesulfonate (Tokyo Chemical Industry Co., Ltd.), Ionic Liquid [7] in which the contents of the 26 elements as determined by ICP-MS measurement were all 10 ppb or less was obtained in the same way as in Example 2-1.

### Example 2-6

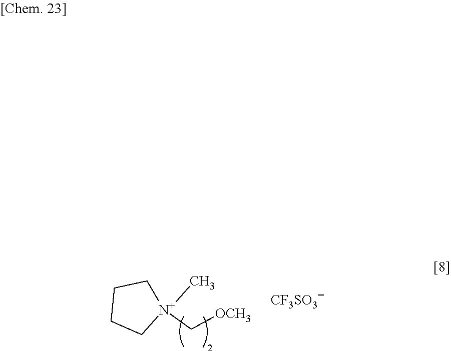

Aside from changing one of the starting materials from dimethyl sulfate to methyl trifluoromethanesulfonate, Ionic Liquid [8] in which the contents of the 26 elements as determined by ICP-MS measurement were all 10 ppb or less was obtained in the same way as in Example 2-2.

### Example 2-7

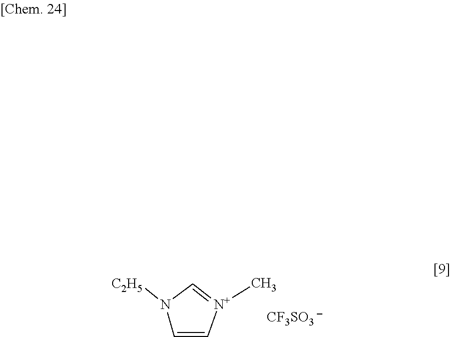

Aside from changing the starting materials from N,N-diethyl-N-2-methoxyethylamine to 1-ethylimidazole (Kanto Chemical Co., Ltd.) and from dimethyl sulfate to methyl trifluoromethanesulfonate, Ionic Liquid [9] in which the contents of the 26 elements as determined by ICP-MS measurement were all 10 ppb or less was obtained in the same way as in Example 2-1.

### Example 2-8

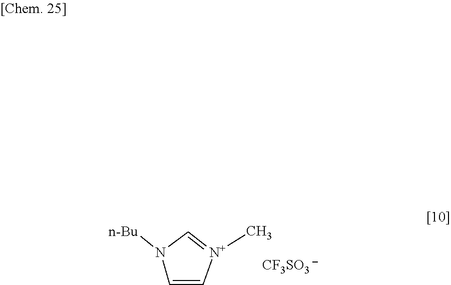

Aside from changing one of the starting materials from 1-ethylimidazole to 1-butylimidazole, Ionic Liquid in which the contents of the 26 elements as determined by ICP-MS measurement were all 10 ppb or less was obtained in the same way as in Example 2-7.

### Example 2-9

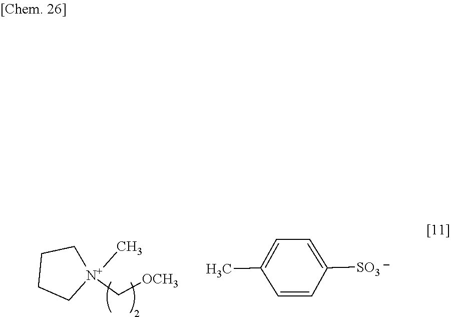

A 3.47 g amount of N-2-methoxyethylpyrrolidine synthesized by an existing method and 5.00 g of methyl p-toluenesulfonate (FUJIFILM Wako Pure Chemical Corporation) were mixed together, and then reacted under a stream of nitrogen at 80° C. for 1 hour. The N-2-methoxyethylpyrrolidine and methyl p-toluenesulfonate used here were each subjected to precise distillation beforehand. Next, 3 mL of precisely distilled acetonitrile was added to this reaction system, which was stirred to give a uniform solution. This solution was added dropwise to 85 mL of precisely distilled toluene and the target substance was separated out as a viscous liquid-like re-precipitate. The solvent was removed from this re-precipitate by decantation and additional solvent was removed from re-precipitate remaining in the vessel by applying a vacuum, giving 7.30 g (yield, 86%) of Ionic Liquid [11]. When left to stand at room temperature, this ionic liquid became a solid (melting point, 53° C.).

A 5.0 g amount of Ionic Liquid was dissolved in 5.0 g of acetonitrile (LC/MS grade, Honeywell). This solution was passed through a plastic column (eluant, acetonitrile) packed with 5.0 g (7.5 mL) of the chelating resin ORLITE DS-22 (Organo Corporation). The resulting eluate was collected in a sterilized polypropylene centrifuge tube “mini” (IWAKI) container and concentrated by applying a vacuum. The concentrate was subjected to ICP-MS measurement, as a result of which the contents of the 26 elements were all 10 ppb or less.

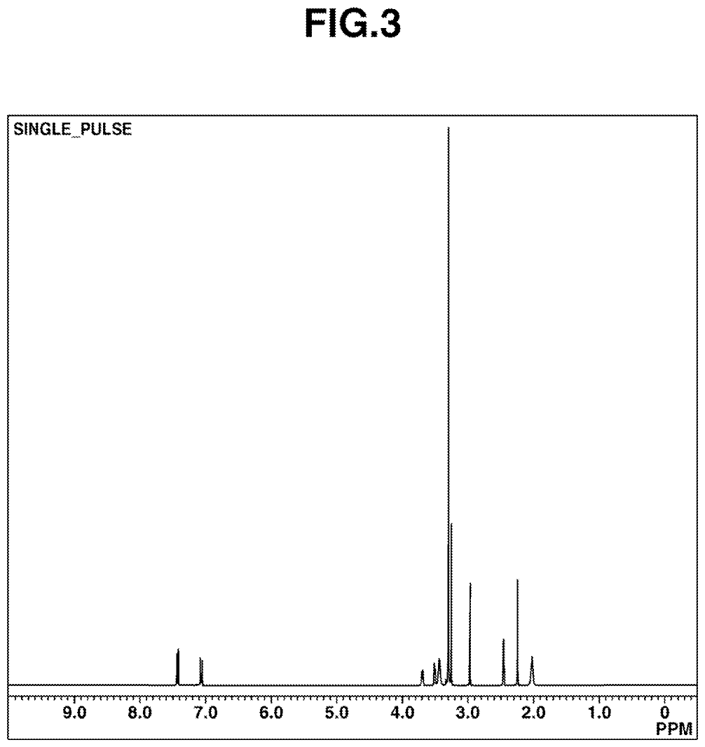

FIG. 3 shows the ¹H-NMR spectrum (solvent, heavy dimethylsulfoxide) of Ionic Liquid [11].

### Example 2-10

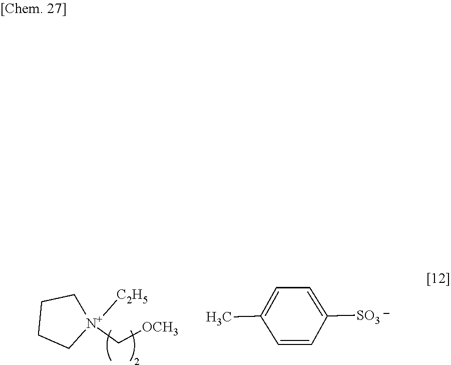

A 3.47 g amount of N-2-methoxyethylpyrrolidine synthesized by an existing method and 5.00 g of ethyl p-toluenesulfonate (Toyokasei Co., Ltd.) were mixed together, and then reacted under a stream of nitrogen at 80° C. for 1.5 hours. The N-2-methoxyethylpyrrolidine and ethyl p-toluenesulfonate used here were each subjected to precise distillation beforehand. Next, 3 mL of precisely distilled acetonitrile was added to this reaction system, which was stirred to give a uniform solution. This solution was added dropwise to 85 mL of precisely distilled toluene and the target substance was separated out as a viscous liquid-like re-precipitate. The solvent was removed from this re-precipitate by decantation and additional solvent was removed from re-precipitate remaining in the vessel by applying a vacuum, giving 7.19 g (yield, 87%) of Ionic Liquid [12]. When left to stand at room temperature, this ionic liquid became a solid (melting point, 54° C.).

A 5.0 g amount of Ionic Liquid was dissolved in 5.0 g of acetonitrile (LC/MS grade, Honeywell). This solution was passed through a plastic column (eluant, acetonitrile) packed with 5.0 g (7.5 mL) of the chelating resin ORLITE DS-22 (Organo Corporation). The resulting eluate was collected in a sterilized polypropylene centrifuge tube “mini” (IWAKI) container and concentrated by applying a vacuum. The concentrate was subjected to ICP-MS measurement, as a result of which the contents of the 26 elements were all 10 ppb or less.

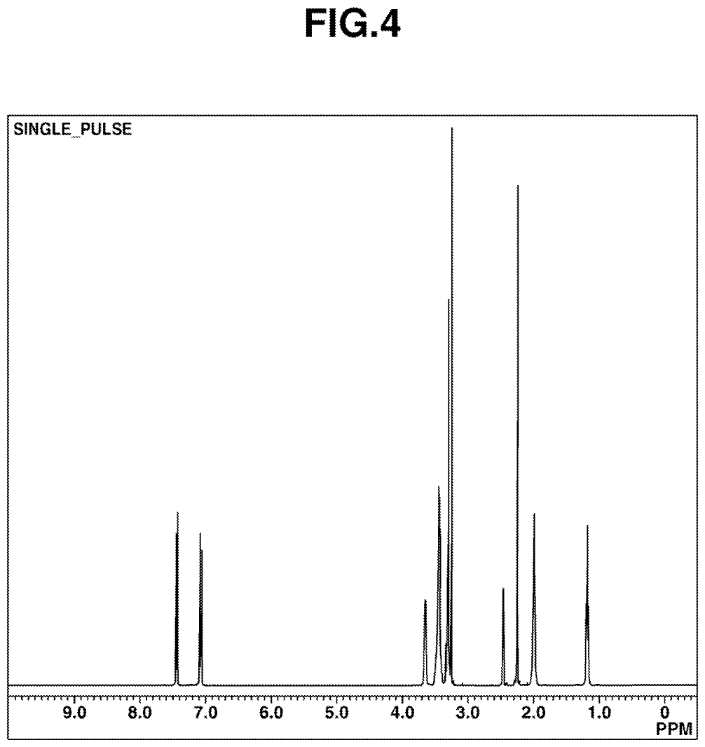

FIG. 4 shows the 1H-NMR spectrum (solvent, heavy dimethylsulfoxide) of Ionic Liquid [12].

### [3] Neutralization Process

### Example 3-1

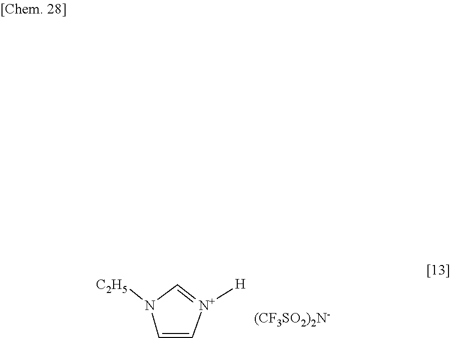

The following were prepared beforehand: 1-ethylimidazole (Kanto Chemical Co., Inc.) which was precisely distilled and subsequently treated with the same weight of the chelating resin ORLITE DS-22, and a solution of 1,1,1-trifluoro-N-[(trifluoromethyl) sulfonyl] methanesulfonamide (Kanto Chemical Co., Inc.) dissolved in the same weight of semiconductor grade methanol, which solution was treated with the same weight of the chelating resin ORLITE DS-22.

These were mixed together so as to make the 1-ethyl-3-methylimidazole and the 1,1,1-trifluoro-N-[(trifluoromethyl) sulfonyl] methanesulfonamide equimolar and stirred for 1 hour. The pH was confirmed to be neutral, after which Ionic Liquid from which methanol was removed by distillation in vacuo was obtained. The contents of the 26 elements in this Ionic Liquid as determined by ICP-MS measurement were all 10 ppb or less.

### Example 3-2

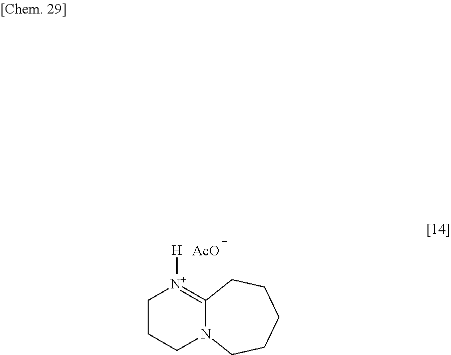 [SMILES: "AcO" ; C1CCC2=[NH+]CCCN2CC1]

1,8-Diazabicyclo [5.4.0]-7-undecene (Tokyo Chemical Industry Co., Ltd.) and acetic acid (Kanto Chemical Co., Inc.) that had each been precisely distilled beforehand and then treated with the same respective weights of the chelating resin ORLITE DS-22 were mixed together within a Teflon®-coated vessel so as to be equimolar, and stirred for 3 hours under heating up to 80° C. The system was then treated for 3 hours under a vacuum, and the unreacted starting materials were removed, giving Ionic Liquid [12]. Ionic Liquid was a liquid at 80° C., but became a solid after being left to cool to room temperature. The contents of the 26 elements in this solid Ionic Liquid as determined by ICP-MS measurement were all 10 ppb or less.

### Example 3-3

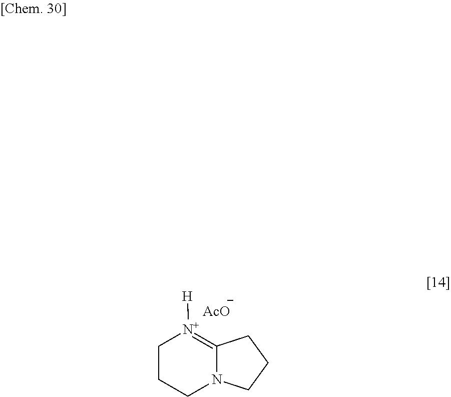

Aside from changing one of the starting materials from 1,8-diazabicyclo [5.4.0]-7-undecene to 1,5-diazabicyclo[4.3.0]-5-nonene, Ionic Liquid in which the contents of the 26 elements as determined by ICP-MS measurement were all 10 ppb or less was obtained in the same way as in Example 3-2. This Ionic Liquid was a liquid at 80° C. but became a solid at room temperature.

1. An ionic liquid comprising cations and anions, wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less.

2. The ionic liquid of claim 1, wherein metal ions of said 16 elements are all included in respective amounts of 10 ppb or less.

3. The ionic liquid of claim 1, wherein metal ions of the 26 elements indicated by the symbols Li, Na, Ca, Mg, Al, K, Ti, Cr, Mn, Fe, Co, Ni, Cu, Zn, Ba, W, V, Sr, Zr, Mo, Ag, Cd, Ta, Tl, Pb and Bi are all included in respective amounts of 100 ppb or less.

4. The ionic liquid of claim 3, wherein metal ions of said 26 elements are all included in respective amounts of 10 ppb or less.

5. The ionic liquid of claim 1, wherein the cations are monovalent cations selected from the group consisting of quaternary ammonium ions, pyridinium ions, cyclic amidinium ions and quaternary phosphonium ions.

6. The ionic liquid of claim 1, wherein the anions are monovalent anions selected from the group consisting of trialkylsilyl group-containing alkyl sulfonate ions, tetrafluoroborate ion, alkyl sulfate ions, hexafluorophosphate ion, bis (trifluoromethanesulfonyl) amide ion, bis (fluorosulfonyl) amide ion, alkyl sulfonate ions, aryl sulfonate ions, trifluoromethanesulfonate ion, acetate ion and alkyl phosphate ions.

7. The ionic liquid of claim 1 being used in a semiconductor fabrication process.

8. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method comprises the step of contacting an ionic liquid composed of cations and anions with a chelating resin material.

9. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method comprises the step of alkylating a tertiary alkylamine or an N-alkylimidazole with a dialkyl sulfate, an alkyl sulfonate, an aryl sulfonate, an alkyl trifluoromethanesulfonate or a trialkyl phosphate.

10. The ionic liquid production method of claim 9, which method comprises the step of using, as the tertiary alkylamine or N-alkylimidazole and as the dialkyl sulfate, alkyl sulfonate, aryl sulfonate, alkyl trifluoromethanesulfonate, or trialkyl phosphate, compounds which contain metal ions of the 16 elements indicated by the symbols Li, Na, Ca, Mg, Al, K, Ti, Cr, Mn, Fe, Co, Ni, Cu, Zn, Ba and W in respective amounts of 100 ppb or less.

11. A method for producing, by neutralization of an acid and a base, an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method comprises the step of using, as the acid and the base, compounds which contain metal ions of said 16 elements in respective amounts of 100 ppb or less.

12. A method for producing an ionic liquid wherein metal ions of the 16 elements indicated by the symbols Li, Na, Ca, Mg, Al, K, Ti, Cr, Mn, Fe, Co, Ni, Cu, Zn, Ba and W are all included in respective amounts of 100 ppb or less, which method comprises the steps of, in order: contacting a salt of a quaternary cation with a chelating resin material so as to remove metal ions included within the salt, converting the anion of the resulting quaternary cation salt to a hydroxide ion by means of an anion exchange resin, and mixing together equivalent amounts of the salt converted to a hydroxide ion and a proton adduct of an anion that gives the desired ionic liquid to effect neutralization.

13. The ionic liquid production method of claim 8, wherein metal ions of said 16 elements are all included in respective amounts of 10 ppb or less.

14. The ionic liquid production method of claim 8, wherein metal ions of the 26 elements indicated by the symbols Li, Na, Ca, Mg, Al, K, Ti, Cr, Mn, Fe, Co, Ni, Cu, Zn, Ba, W, V, Sr, Zr, Mo, Ag, Cd, Ta, Tl, Pb and Bi are all included in respective amounts of 100 ppb or less.

15. The ionic liquid production method of claim 14, wherein metal ions of said 26 elements are all included in respective amounts of 10 ppb or less.

16. An ionic liquid of formula (A) below 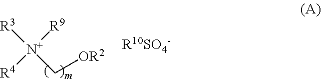
(wherein R³, R⁴ and R⁹ are each independently an alkyl group of 1 to 3 carbon atoms, R¹⁰ is an alkyl group of 1 to 4 carbon atoms, R² is a methyl group or an ethyl group, and m is the integer 1 OF 2).

17. The ionic liquid according to claim 16 of formula (A1) below 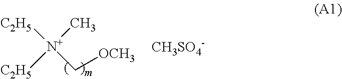
(wherein m is the integer 1 or 2).

18. The ionic liquid of formula (B) below 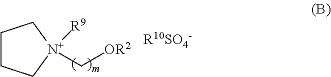
(wherein R⁹ is an alkyl group of 1 to 3 carbon atoms, R² is a methyl group or an ethyl group, R¹⁰ is an alkyl group of 1 to 4 carbon atoms, and m is the integer 1 or 2).

19. The ionic liquid according to claim 18 of formula (B1) below 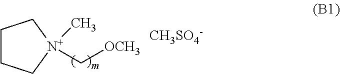
(wherein m is the integer 1 or 2).

20. The ionic liquid of formula (C) below 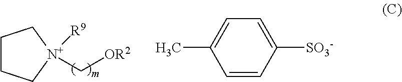
(wherein R⁹ is an alkyl group of 1 to 3 carbon atoms, R² is a methyl group or an ethyl group, and m is the integer 1 or 2).

21. The ionic liquid of claim 20, wherein R² is a methyl group and R⁹ is a methyl group or an ethyl group.
